(19) United States
(12) Patent Application Publication
(10) Pub. No.: US 2025/0009343 A1
(43) Pub. Date: Jan. 9, 2025

(54) ULTRASOUND DIAGNOSIS APPARATUS, ULTRASOUND DIAGNOSIS METHOD, AND IMAGE PROCESSING APPARATUS

(71) Applicant: CANON MEDICAL SYSTEMS CORPORATION, Tochigi (JP)

(72) Inventors: Hiroki TAKAHASHI, Nasushiobara (JP); Tetsuya YOSHIDA, Nasushiobara (JP)

(73) Assignee: CANON MEDICAL SYSTEMS CORPORATION, Tochigi (JP)

(21) Appl. No.: 18/760,080

(22) Filed: Jul. 1, 2024

(30) Foreign Application Priority Data
Jul. 3, 2023 (JP) 2023-109594

(51) Int. Cl. A61B 8/08; A61B 8/00; A61B 8/06; G16H 50/20

(52) U.S. Cl. CPC A61B 8/5223; A61B 8/06; A61B 8/0866; A61B 8/463; A61B 8/488; G16H 50/20

(57) ABSTRACT

An ultrasound diagnosis apparatus according to an embodiment includes processing circuitry. The processing circuitry includes: transmitter/receiver circuitry configured to transmit an ultrasound wave to the inside of an examined subject and to receive an echo signal emitted in response to the ultrasound wave; and processing circuitry configured to extract, with respect to each spatial point, information that is related to motion velocity of a target inside the examined subject and included in a signal data sequence obtained from the echo signal, to calculate periodicity information of a movement of the examined subject on the basis of the information, and to generate a spatial distribution of the periodicity information.

### CROSS-REFERENCE TO RELATED APPLICATIONS

This application is based upon and claims the benefit of priority from Japanese Patent Application No. 2023-109594, filed on Jul. 3, 2023; the entire contents of which are incorporated herein by reference.

### FIELD

Embodiments described herein relate generally to an ultrasound diagnosis apparatus, an ultrasound diagnosis method, and an image processing apparatus.

### BACKGROUND

Ultrasound diagnosis apparatuses have installed therein a large number of functions for blood flow imaging using the Doppler phenomenon. A conventional ultrasound diagnosis apparatus is capable, for example, of displaying the velocity and the orientation of a blood flow or a blood flow echo intensity.

Further, if it becomes possible to express, in an image, periodicity of chronological changes in hemodynamics such as, for example, a pattern of chronological changes in approximately one heartbeat, it is expected that there will be various useful clinical applications, such as identifying blood flows of a mother and a fetus or evaluating changes in renal hemodynamics relevant to a heart disease.

### BRIEF DESCRIPTION OF THE DRAWINGS

FIG. 1 is a drawing illustrating an example of an ultrasound diagnosis apparatus according to an embodiment;

FIG. 2 is a drawing for explaining a background of the embodiment;

FIG. 3 is a flowchart illustrating an example of processes performed by an ultrasound diagnosis apparatus according to a first embodiment;

FIG. 4 is a drawing for explaining a configuration of an experiment performed by the ultrasound diagnosis apparatus according to the embodiment;

FIG. 5 is another drawing for explaining the configuration of the experiment performed by the ultrasound diagnosis apparatus according to the embodiment;

FIG. 6 is a drawing illustrating an example of data obtained by the ultrasound diagnosis apparatus according to the embodiment;

FIG. 7 is a drawing illustrating another example of data obtained by the ultrasound diagnosis apparatus according to the embodiment;

FIG. 8 is a flowchart illustrating an example of processes performed by an ultrasound diagnosis apparatus according to a second embodiment;

FIG. 9 is a flowchart illustrating an example of processes performed by an ultrasound diagnosis apparatus according to a third embodiment; and

FIG. 10 is a flowchart illustrating an example of processes performed by an ultrasound diagnosis apparatus according to a fourth embodiment.

### DETAILED DESCRIPTION

An ultrasound diagnosis apparatus according to an embodiment includes processing circuitry. The ultrasound diagnosis apparatus includes: transmitter/receiver circuitry configured to transmit an ultrasound wave to the inside of an examined subject and to receive an echo signal emitted in response to the ultrasound wave; and processing circuitry configured to extract, with respect to each spatial point, information that is related to motion velocity of a target inside the examined subject and included in a signal data sequence obtained from the echo signal, to calculate periodicity information of a movement of the examined subject on the basis of the information, and to generate a spatial distribution of the periodicity information.

### First Embodiment

Embodiments of an ultrasound diagnosis apparatus, an ultrasound diagnosis method, and an image processing apparatus will be explained in detail below, with reference to the accompanying drawings.

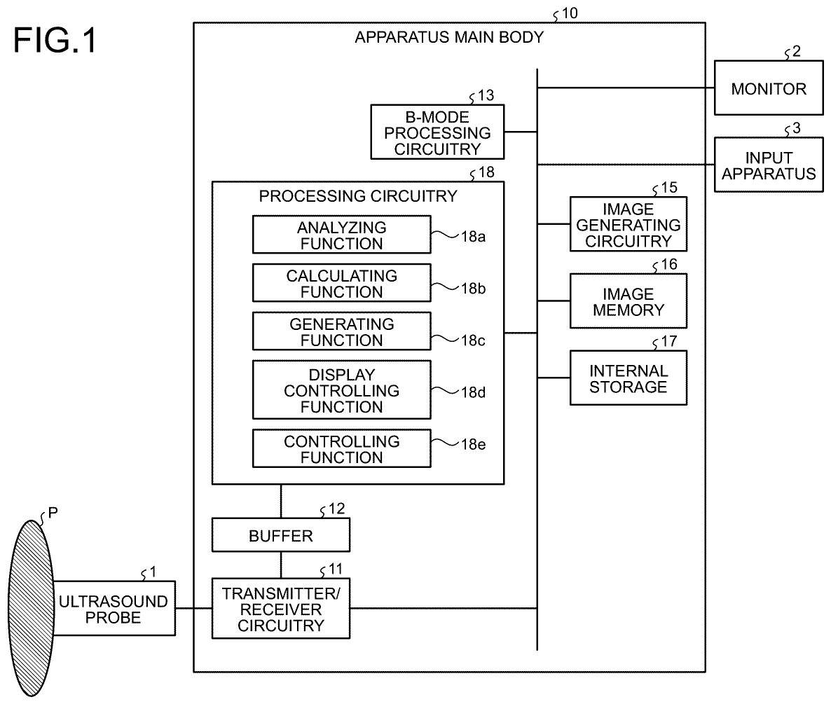

To begin with, a configuration of an ultrasound diagnosis apparatus according to an embodiment will be explained. FIG. 1 is a block diagram illustrating an exemplary configuration of the ultrasound diagnosis apparatus according to the embodiment. As illustrated in FIG. 1, the ultrasound diagnosis apparatus according to the embodiment includes an ultrasound probe 1, a monitor 2, an input apparatus 3, and an apparatus main body 10.

The ultrasound probe 1 is connected to the apparatus main body 10 for transmitting and receiving an ultrasound wave. The ultrasound probe 1 includes a plurality of piezoelectric transducer elements, for example. The plurality of piezoelectric transducer elements are configured to generate the ultrasound wave on the basis of a drive signal supplied from transmitter/receiver circuitry 11 included in the apparatus main body 10. For example, the plurality of piezoelectric transducer elements are configured to transmit, to an examined subject (hereinafter, “patient”) P, the ultrasound wave having an intensity corresponding to the magnitude of voltage applied from the transmitter/receiver circuitry 11. Further, the plurality of piezoelectric transducer elements included in the ultrasound probe 1 are configured to receive a reflected wave (an echo) from the patient P, to convert the received reflected wave into an electrical signal (a reflected-wave signal), and to transmit the reflected-wave signal to the apparatus main body 10. Also, the ultrasound probe 1 includes a matching layer provided for the piezoelectric transducer elements, a backing member configured to prevent the ultrasound wave from propagating rearward from the piezoelectric transducer elements, and the like. In this situation, the ultrasound probe 1 is detachably connected to the apparatus main body 10.

When the ultrasound wave is transmitted from the ultrasound probe 1 to the patient P, the transmitted ultrasound wave is repeatedly reflected on a surface of discontinuity of acoustic impedances at a tissue in the body of the patient P and is received as the reflected wave by the plurality of piezoelectric transducer elements included in the ultrasound probe 1. The amplitude of the received reflected wave is dependent on the difference between the acoustic impedances on the surface of discontinuity on which the ultrasound wave is reflected.

In this situation, the embodiment is applicable to the situations where the ultrasound probe 1 is a one-dimensional (1D) array probe configured to two-dimensionally scan the patient P or is a mechanical four-dimensional (4D) probe or a two-dimensional (2D) array probe configured to three-dimensionally scan the patient P.

The input apparatus 3 is realized by using a mouse, a keyboard, a button, a panel switch, a touch command screen, a foot switch, a trackball, a joystick, and/or the like. The input apparatus 3 is configured to receive various types of setting requests from an operator of the ultrasound diagnosis apparatus and to transfer the received various types of setting requests to the apparatus main body 10.

The monitor 2 is configured to display a Graphical User Interface (GUI) used by the operator of the ultrasound diagnosis apparatus for inputting the various types of setting requests via the input apparatus 3 and to display an ultrasound image represented by ultrasound image data generated in the apparatus main body 10, and the like. The monitor 2 is realized by using a liquid crystal monitor, a Cathode Ray Tube (CRT) monitor, or the like.

The apparatus main body 10 is an apparatus configured to generate the ultrasound image data on the basis of the reflected-wave signals transmitted thereto from the ultrasound probe 1. The apparatus main body 10 illustrated in FIG. 1 is an apparatus capable of generating two-dimensional ultrasound image data on the basis of a two-dimensional reflected-wave signal and capable of generating three-dimensional ultrasound image data on the basis of a three-dimensional reflected-wave signal.

As illustrated in FIG. 1, the apparatus main body 10 includes the transmitter/receiver circuitry 11, a buffer 12, B-mode processing circuitry 13, image generating circuitry 15, an image memory 16, internal storage 17, and processing circuitry 18.

The transmitter/receiver circuitry 11 is configured to control an ultrasound scan performed by the ultrasound probe 1, on the basis of an instruction from the processing circuitry 18 (explained later). In this situation, the ultrasound scan denotes ultrasound transmission/reception, for example. The transmitter/receiver circuitry 11 includes a pulse generator, transmission delay circuitry, a pulser, and the like and is configured to supply the drive signal to the ultrasound probe 1. The pulse generator is configured to repeatedly generate a rate pulse used for forming a transmission ultrasound wave at a prescribed Pulse Repetition Frequency (PRF). Further, the transmission delay circuitry is configured to apply, to the rate pulses generated by the pulse generator, delay periods that respectively correspond to the piezoelectric transducer elements and are necessary for converging the ultrasound wave generated by the ultrasound probe 1 into the form of a beam and determining transmission directionality. Further, the pulser is configured to apply the drive signal to the ultrasound probe 1, with timing based on the rate pulses. In other words, the transmission delay circuitry is configured to arbitrarily adjust transmission directions of the ultrasound wave transmitted from surfaces of the piezoelectric transducer elements, by varying the delay periods applied to the rate pulses.

For example, under control of the processing circuitry 18, the transmitter/receiver circuitry 11 is configured to cause the ultrasound probe 1 to perform the ultrasound scan by which data sequences between frames are acquired as a doppler data sequence. In an example, the ultrasound probe 1 and the transmitter/receiver circuitry 11 are configured to acquire a data sequence of reflected-wave data in a certain position, by acquiring the reflected-wave data from the certain position over a plurality of frames along the time direction.

Further, the transmitter/receiver circuitry 11 includes amplifier circuitry, an Analog/Digital (A/D) converter, reception delay circuitry, an adder, quadrature detection circuitry, and the like and is configured to generate the reflected-wave data by performing various types of processes on the reflected-wave signals transmitted thereto from the ultrasound probe 1. Further, the transmitter/receiver circuitry 11 is configured to store the generated reflected-wave data into the buffer 12. The amplifier circuitry is configured to amplify the reflected-wave signal with respect to each channel and to perform a gain correction process. The A/D converter is configured to perform an A/D conversion on the gain-corrected reflected-wave signals. The reception delay circuitry is configured to apply, to the digital data, a reception delay period necessary for determining reception directionality. The adder is configured to perform a process of adding together the reflected-wave signals to which the reception delay period was applied by the reception delay circuitry. As a result of the adding process by the adder, reflected components from a direction corresponding to the reception directionality of the reflected-wave signals are emphasized. In this situation, the process of adjusting a phase with the reception delay with respect to each of the reflected-wave signals of the elements and adding the results together may be referred to as a phased addition process or a beam forming process.

The quadrature detection circuitry is configured to convert an output signal from the adder into an In-phase signal (an I signal) and a Quadrature-phase signal (a Q signal) in a baseband. After that, the quadrature detection circuitry is configured to store the I signal and the Q signal (hereinafter, “IQ signals”) into the buffer 12, as the reflected-wave data.

The buffer 12 is a memory configured to temporarily store therein the reflected-wave data generated by the transmitter/receiver circuitry 11. More specifically, the buffer 12 is configured to store therein the reflected-wave data corresponding to a number of frames or the reflected-wave data corresponding to a number of volumes. For example, the buffer 12 may be a First-In/First-Out (FIFO) memory and is configured to store therein the reflected-wave data corresponding to a prescribed number of frames under control of the transmitter/receiver circuitry 11. Further, for example, when reflected-wave data corresponding to one frame is newly generated by the transmitter/receiver circuitry 11, the buffer 12 is configured, under the control of the transmitter/receiver circuitry 11, to discard reflected-wave data corresponding to another frame that was generated earliest and to store therein the newly-generated reflected-wave data corresponding to the one frame. For example, the buffer 12 is realized by using a semiconductor memory element such as a Random Access Memory (RAM), a flash memory, or the like.

The B-mode processing circuitry 13 and the processing circuitry 18 are signal processing units configured to perform various types of signal processing processes on the reflected-wave data generated by the transmitter/receiver circuitry 11 from the reflected-wave signals. The B-mode processing circuitry 13 and the processing circuitry 18 are realized by using processors, for example. The B-mode processing circuitry 13 is configured to generate data (B-mode data) in which the signal intensity at each of a plurality of sampling points is expressed with a brightness level by reading the reflected-wave data from the buffer 12 and further performing a logarithmic amplifying process, an envelope detection process, a logarithmic compression, and/or the like on the read reflected-wave data.

The processing circuitry 18 includes an analyzing function 18a, a calculating function 18b, a generating function 18c, a display controlling function 18d, and a controlling function 18e. In this situation, processing functions executed by the processing circuitry 18 including the analyzing function 18a, the calculating function 18b, the generating function 18c, the display controlling function 18d, and the controlling function 18e are recorded in the internal storage 17 in the form of computer-executable programs. The processing circuitry 18 is configured to read and execute the programs from the internal storage 17 so as to realize the functions corresponding to the read programs. In other words, the processing circuitry 18 that has read the programs has the functions illustrated within the processing circuitry 18 in FIG. 1.

Various types of processes performed by the analyzing function 18a, the calculating function 18b, the generating function 18c, the display controlling function 18d, and the controlling function 18e will be explained later. In this situation, the analyzing function 18a, the calculating function 18b, the generating function 18c, the display controlling function 18d, and the controlling function 18e are examples of an analyzing unit, a calculating unit, a generating unit, a display controlling unit, and a controlling unit, respectively. Further, the transmitter/receiver circuitry 11 and the monitor 2 are examples of a transmitter/receiver unit and a display unit, respectively.

By employing the analyzing function 18a, the processing circuitry 18 is configured to read the reflected-wave data from the buffer 12 and to further perform a frequency analysis on the read reflected-wave data, so as to estimate motion information based on the Doppler effect of a moving member within a scanned range and to generate data (Doppler data) representing the estimated motion information. For example, as the motion information of the moving member, the processing circuitry 18 is configured to estimate an average velocity value, an average dispersion value, an average power value, and the like with respect to each of multiple sampling points and to generate the Doppler data representing the estimated motion information. In this situation, the moving member may be, for example, a blood flow, a tissue such as a cardiac wall, or a contrast agent.

By employing a function of the analyzing function 18a described above, the ultrasound diagnosis apparatus according to the present embodiment is capable of implementing a color Doppler method which may also be referred to as a Color Flow Mapping (CFM) method. According to the CFM method, the ultrasound transmission/reception is performed multiple times on a plurality of scanning lines. Further, according to the CFM method, a blood flow signal derived from a blood flow is extracted, while suppressing signals (clutter signals) derived from motionless tissues or slow-moving tissues, by applying a Moving Target Indicator (MTI) filter to a data sequence corresponding to mutually the same position. For example, when the data sequence of the reflected-wave data corresponding to mutually the same position is input to the MTI filter, the MTI filter is configured to output a blood flow signal in which the blood flow component is dominant while the clutters are suppressed. As the MTI filter, for example, it is possible to use an adaptive principal component analysis filter configured to vary a coefficient in response to an input signal.

Further, by employing the calculating function 18b, the processing circuitry 18 is configured to calculate periodicity information of movements of the patient. By employing the generating function 18c, the processing circuitry 18 is configured to generate a spatial distribution of the periodicity information. Details of the calculating function 18b and the generating function 18c will be explained later.

Further, by employing the display controlling function 18d, the processing circuitry 18 is configured to exercise display control on the monitor 2.

By employing the controlling function 18e, the processing circuitry 18 is configured to control overall processes performed by the ultrasound diagnosis apparatus. More specifically, the processing circuitry 18 is configured to control processes performed by the transmitter/receiver circuitry 11, the B-mode processing circuitry 13, Doppler processing circuitry 14, and the image generating circuitry 15, on the basis of the various types of setting requests input by the operator via the input apparatus 3 and various types of control programs and various types of data read from the internal storage 17. For example, by controlling the ultrasound probe 1 via the transmitter/receiver circuitry 11, the processing circuitry 18 is configured to control the ultrasound scan.

The image generating circuitry 15 is configured to generate the ultrasound image data from the data generated by the B-mode processing circuitry 13 and the processing circuitry 18.

In this situation, generally speaking, the image generating circuitry 15 is configured to convert (by performing a scan convert process) a scanning line signal sequence from an ultrasound scan into a scanning line signal sequence in a video format used by television, for example, and to generate display-purpose ultrasound image data. More specifically, the image generating circuitry 15 is configured to generate the display-purpose ultrasound image data by performing a coordinate transformation process compliant with the ultrasound scanning mode used by the ultrasound probe 1. Further, as various types of image processing processes besides the scan convert process, the image generating circuitry 15 is configured to perform, for example, an image processing process (a smoothing process) to re-generate an average brightness value image, an image processing process (an edge enhancement process) that uses a differential filter inside an image, or the like, by using a plurality of image frames resulting from the scan convert process. Also, the image generating circuitry 15 is configured to combine text information of various types of parameters, scale graduations, body marks, and the like with the ultrasound image data.

The image memory 16 is a memory configured to store therein the display-purpose image data generated by the image generating circuitry 15. Further, the image memory 16 is also capable of storing therein any of the data generated by the B-mode processing circuitry 13 and the processing circuitry 18.

The internal storage 17 is configured to store therein control programs for performing the ultrasound transmission/reception, image processing processes, and display processes, as well as various types of data such as diagnosis information (e.g., patient IDs, medical doctors' observations, etc.), diagnosis protocols, and various types of body marks. Further, the internal storage 17 may also be used for saving any of the image data stored in the image memory 16, as necessary. Further, the data stored in the internal storage 17 may be transferred to an external apparatus via an interface (not illustrated). In addition, the internal storage 17 is also capable of storing therein data transferred thereto from an external apparatus via an interface (not illustrated). For example, the internal storage 17 is realized by using a semiconductor memory element such as a flash memory, or a hard disk, an optical disk, or the like.

Further, for example, an image processing apparatus according to the embodiment may be structured by using the image generating circuitry 15, the image memory 16, the internal storage 17, the monitor 2, and the input apparatus 3. The image processing apparatus according to the embodiment may be incorporated in the ultrasound diagnosis apparatus or may be configured as an image processing apparatus independent of the ultrasound diagnosis apparatus.

Next, a background of the embodiment will be explained. Ultrasound diagnosis apparatuses have installed therein a large number of functions for blood flow imaging using the Doppler phenomenon. More specifically, a conventional ultrasound diagnosis apparatus is capable of displaying the velocity and the orientation of a blood flow or a blood flow echo intensity.

The ultrasound diagnosis apparatus according to the embodiment is configured, in addition to the above, to express, in an image, periodicity of chronological changes in hemodynamics, i.e., for example, a pattern of chronological changes in approximately one heartbeat. It is considered that the capability to express, in an image, the periodicity of the chronological changes in hemodynamics is useful, for example, for identifying blood flows of a mother and a fetus or for evaluating changes in renal hemodynamics relevant to a heart disease.

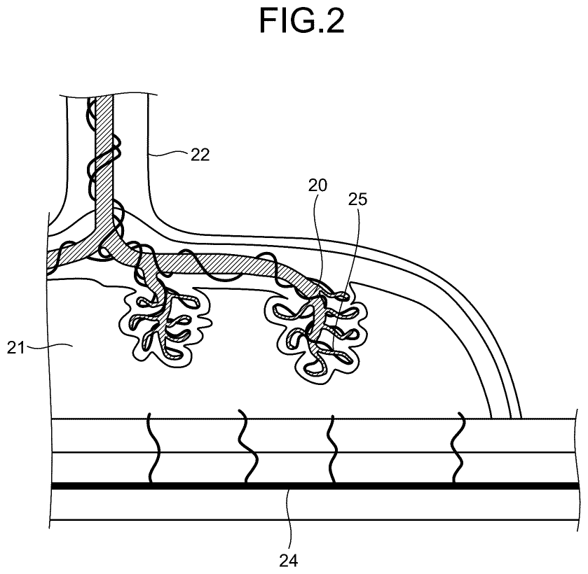

A first application example of imaging the periodicity of the chronological changes in hemodynamics addresses, for example, identifying blood flows of a mother and a fetus. FIG. 2 schematically illustrates fetal blood vessels and a maternal blood flow in the placenta. Umbilical blood vessels 20 are the fetal blood vessels and are connected to the fetus via umbilical cord 22, so that oxygen and nutrition are obtained via chorionic villi 25 from intervillous space 21 being a maternal blood pool. In this situation, for evaluating functions of the placenta, it is important that the chorionic villi 25 are sufficiently structured (the fetal blood vessels) and that blood vessels branched from uterine artery 24 sufficiently reach the intervillous space 21 (maternal blood vessels). In order to confirm these items, it is necessary to identify whether each of the small blood vessels present in the intervillous space 21 and the surroundings thereof is a maternal blood vessel or a fetal blood vessel. However, because the umbilical blood vessels 20, the chorionic villi 25, and the maternal blood flow are adjacent to one another, it would be difficult to identify the blood flows of the mother and the fetus.

Accordingly, in the ultrasound diagnosis apparatus according to the embodiment, the processing circuitry 18 is configured to calculate the periodicity information of the movements of the patient and to generate the spatial distribution of the periodicity information. In this situation, in the umbilical blood vessels 20, blood circulates on the basis of a cycle of pulsation of the fetal heart. In contrast, in the maternal blood vessels, blood circulates on the basis of a cycle of pulsation of the mother's heart. As a result, the frequencies of the movements are different between the fetal blood vessels and the maternal blood vessels. Consequently, as a result of the processing circuitry 18 calculating the periodicity information of the movements of the patient and generating the spatial distribution of the periodicity information, it is possible to identify the blood flows of the mother and the fetus.

A second application example of imaging the periodicity of the chronological changes in hemodynamics addresses, for example, identifying a pattern of chronological changes in renal hemodynamics, for the purpose of evaluating worsening of the renal hemodynamics relevant to a heart disease i.e., a cardiorenal syndrome. In particular, it is known that blood flow patterns of intrarenal interlobar veins have a strong correlation with prognoses of heart failure. More specifically, it has been reported that blood flow patterns of interlobar veins of heart disease patients have a single phase or two phases and that prognoses of cases with a single phase are worse than those of cases with two phases.

Accordingly, because the processing circuitry 18 is configured to calculate the periodicity information of the chronological changes in a blood flow in an interlobar vein, it is expected to be possible to use the calculation result for predicting a prognosis of heart failure.

In view of the objectives described above, the ultrasound diagnosis apparatus according to the present embodiment includes the transmitter/receiver circuitry 11 serving as a transmitter/receiver unit and the processing circuitry 18. The transmitter/receiver circuitry 11 is configured to transmit an ultrasound wave to the inside of the patient and to receive an echo signal emitted in response to the ultrasound wave. By employing the analyzing function 18a, the processing circuitry 18 is configured to extract, with respect to each spatial point, information that is related to motion velocity of a target inside the patient and included in a signal data sequence obtained from the echo signal. By employing the calculating function 18b, the processing circuitry 18 is configured to calculate periodicity information of movements of the patient on the basis of the information related to the motion velocity. By employing the generating function 18c, the processing circuitry 18 is configured to generate a spatial distribution of the periodicity information. In this situation, the periodicity information is information relevant to a motion cycle of the movements of the patient. Examples thereof include a frequency spectrum obtained by performing a Fourier transform on data obtained with respect to each spatial point and information about an average frequency obtained by averaging the frequency spectra among frequencies.

Further, the ultrasound diagnosis method according to the present embodiment includes: transmitting an ultrasound wave to the inside of a patient and receiving an echo signal emitted in response to the ultrasound wave; extracting, with respect to each spatial point, information that is related to motion velocity of a target inside the patient and included in a signal data sequence obtained from the echo signal; calculating periodicity information of movements of the patient on the basis of the information; and generating a spatial distribution of the periodicity information.

Further, the image processing apparatus according to the embodiment includes the processing circuitry 18. By employing analyzing function 18a, the processing circuitry 18 is configured to extract, with respect to each spatial point, information that is related to motion velocity of a target inside the patient and included in a signal data sequence obtained from an echo signal emitted in response to an ultrasound wave transmitted to the inside of the patient. By employing calculating function 18b, the processing circuitry 18 is configured to calculate periodicity information of movements of the patient on the basis of the information. By employing generating function 18c, the processing circuitry 18 is configured to generate a spatial distribution of the periodicity information.

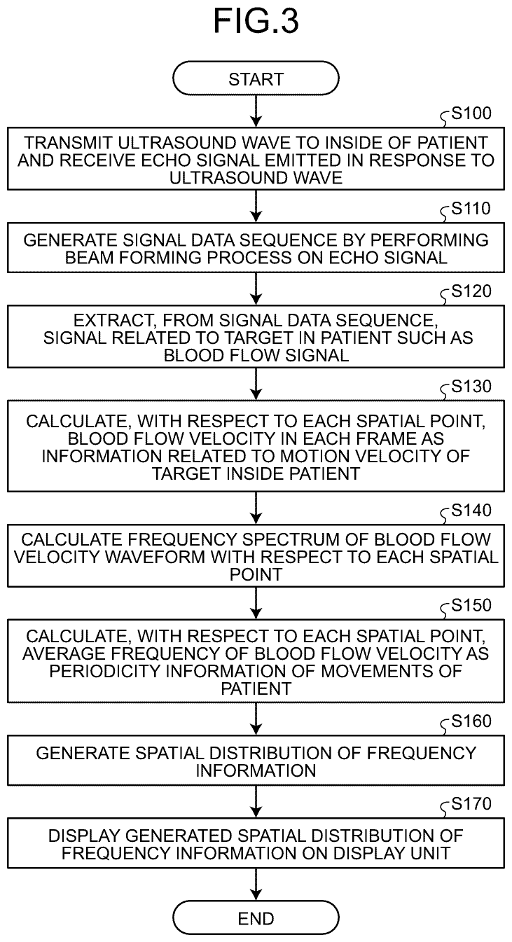

Details of the above processes will be explained, with reference to FIG. 3. FIG. 3 is a flowchart illustrating a flow in the processes performed by the ultrasound diagnosis apparatus according to the first embodiment.

To begin with, at step S100, the transmitter/receiver circuitry 11 serving as a transmitter/receiver unit transmits an ultrasound wave to the inside of the patient and receives an echo signal emitted in response to the ultrasound wave. In an example, the transmitter/receiver circuitry 11 receives echo data over a plurality of frames by using a plane wave compounding scheme. However, possible embodiments are not limited to receiving the echo data by using the plane wave compounding scheme. The transmitter/receiver circuitry 11 may perform the ultrasound transmission/reception by using other schemes for scanning a region of interest at a high framerate, such as a synthetic transmit aperture method that uses a converged ultrasound wave transmission, for example.

Subsequently, at step S110, by employing the analyzing function 18a, the processing circuitry 18 generates a signal data sequence, by performing the beam forming process on the echo signal received at step S100.

After that, at step S120, by employing the analyzing function 18a, the processing circuitry 18 extracts a signal related to the target in the patient, from the signal data sequence generated at step S110. In an example, by employing the analyzing function 18a, the processing circuitry 18 extracts a blood flow signal from the signal data sequence generated at step S110. For example, by employing the analyzing function 18a, the processing circuitry 18 may extract the blood flow signal, by applying the Moving Target Indicator (MTI) filter to the signal data sequence generated at step S110. In another example, by employing the analyzing function 18a, the processing circuitry 18 may extract the blood flow signal by implementing a wall motion filter method on the signal data sequence generated at step S110.

Subsequently, at step S130, by employing the analyzing function 18a serving as an analyzing unit, the processing circuitry 18 extracts, with respect to each spatial point x, information s(t,x) related to motion velocity of the target inside the patient, from the signal data sequence obtained from the echo signal, for each frame t. In an example, by employing the analyzing function 18a, the processing circuitry 18 calculates blood flow velocity from the blood flow signal with respect to each frame, by implementing an autocorrelation method or a Doppler spectrum method. In other words, by employing the analyzing function 18a, the processing circuitry 18 extracts, with respect to each frame t, a time waveform of Doppler velocity from the data sequence obtained on the basis of the echo signal, as the information s(t,x) related to the motion velocity of the target inside the patient, by implementing the autocorrelation method, the Doppler spectrum method, or the like.

After that, at step S140, by employing the calculating function 14b, the processing circuitry 18 calculates a frequency spectrum S(i,x) of the blood flow velocity waveform, with respect to each spatial point x. In this situation, i denotes an index corresponding to the frequency in the frame direction.

In an example, by employing the calculating function 14b, the processing circuitry 18 calculates a complex frequency spectrum S(i,x) with respect to a plurality of values of a frequency f(i), by performing a Fourier analysis on the information s(t,x) related to the motion velocity extracted with respect to each spatial point x. In this situation, f(i) denotes the frequency in the frame direction and expresses a cyclic fluctuation of the motion information of the target that is originating from heartbeats or the like, for example. In the following section, the complex frequency spectrum S(i,x) obtained with respect to each spatial point x will simply be referred to as S(i).

Subsequently, at step S150, by employing the calculating function 14b serving as a calculating unit, the processing circuitry 18 calculates, with respect to each space, periodicity information of the movements of the patient, on the basis of the information related to the motion velocity of the target inside the patient. In an example, by employing the calculating function 14b, the processing circuitry 18 calculates, with respect to each spatial point, an average frequency related to chronological changes in the motion of the target, e.g., an average frequency of the blood flow velocity, as the periodicity information of the movements of the patient.

In an example, by employing the calculating function 14b serving as a calculating unit, the processing circuitry 18 calculates an average frequency μ_{f} as the periodicity information, by adding together values of the frequency f(i) while applying weights thereto with the use of the magnitudes of the frequency spectra S(i) calculated at step S140. In an example, by employing the calculating function 14b, the processing circuitry 18 calculates the average frequency μ_{f} as the periodicity information, by adding together the values of the frequency f(i) while applying the weights thereto with the use of the magnitudes of the frequency spectra S(i) calculated at step S140, according to Expression (1) presented below.

$${μ}_{f} = \frac{{\sum }_{i} f ( i ) \vert S ( i ) \vert}{{\sum }_{i} \vert S ( i ) \vert} \qquad ( 1 )$$

After that, at step S160, by employing the generating function 14c serving as a generating unit, the processing circuitry 18 generates a spatial distribution μ_{f}(x) of frequency information, on the basis of the periodicity information of the movements of the patient such as the average frequency μ_{f} that was calculated at step S150 with respect to each spatial point x.

Subsequently, at step S170, by employing the display controlling function 18d, the processing circuitry 18 causes a display unit to display the generated spatial distribution of the frequency information.

As additional information about individual processes in the application examples, when the purpose is to identify the blood flows of a mother and a fetus, the target inside the patient is a region including the umbilical blood vessels 20. By employing the calculating function 18b, the processing circuitry 18 is configured, with respect to the intervillous space 21 being a region corresponding to the maternal blood vessels, to calculate information corresponding to a maternal heart rate, as periodicity information of a first movement and is configured, with respect to a region corresponding to the umbilical blood vessels 20, to calculate information corresponding to a fetal heart rate as periodicity information of a second movement. For example, by employing the calculating function 18b, the processing circuitry 18 is configured to set threshold values corresponding to the maternal heart rate and to the fetal heart rate, so as to determine that a region in question is the region of maternal blood, when the calculated periodicity information of the movements is within the numerical value range set as the maternal heart rate and that a region in question is the region of the fetal blood vessel when the calculated periodicity information of the movements is within the numerical value range set as the fetal heart rate.

Further, when the purpose is to identify whether the phase is the single phase or the two phases regarding the intrarenal interlobar vein blood flow for a cardiorenal analysis, the target inside the patient is a region including the intrarenal interlobar vein. By employing the calculating function 18b, the processing circuitry 18 is configured to calculate the periodicity information of the blood flow in the intrarenal interlobar vein and to generate information related to a state of a heart disease on the basis of the calculated periodicity information of the blood flow in the intrarenal interlobar vein. In an example, by employing the calculating function 18b, the processing circuitry 18 is configured to determine that there is a high risk that the state of the heart disease may have an adverse prognosis when the cyclic pattern of the blood flow in the intrarenal interlobar vein has a single phase and to determine that there is a relatively low risk that the heart disease may have an adverse prognosis when the cyclic pattern has two phases.

Further, when the purpose is to realize imaging of an intracerebral blood flow or the like, for example, it is possible to realize the blood flow imaging with a high sensitivity, by imaging a spatial distribution of the periodicity information with respect to each of different motion directions of the target, for example. In an example, at step S120, by employing the analyzing function 14a, the processing circuitry 18 may extract a signal included in a signal data sequence with respect to each of the different motion directions of the target and, by employing the calculating function 18b, generate a spatial distribution of the periodicity information with respect to each of the different motion directions of the target.

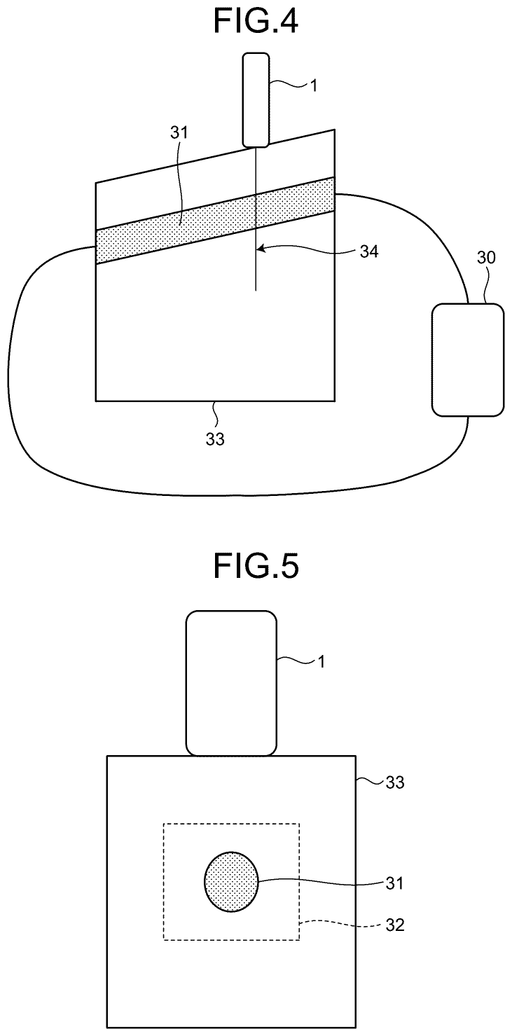

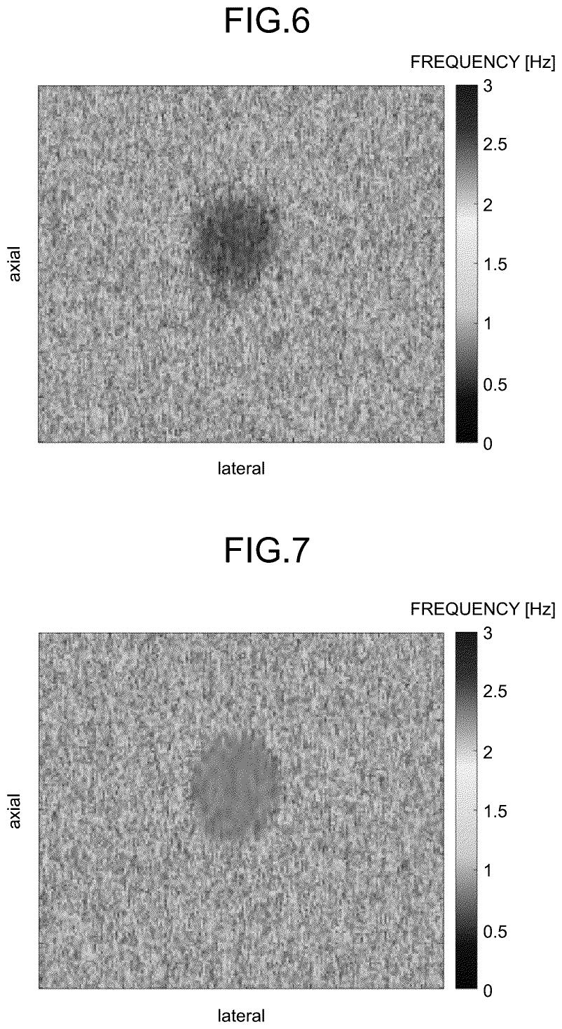

To verify validity of the method according to the first embodiment, we performed an ultrasound measuring experiment by using a phantom containing a dispersed fluid. FIGS. 4 and 5 are schematic drawings illustrating an outline of the ultrasound measuring experiment. FIG. 4 is a cross-sectional view in the long-axis direction of a flow path of a dispersed fluid 31 and illustrates a cross-sectional view in the short-axis direction of the flow path of the dispersed fluid 31. In the ultrasound measuring experiment, by using the ultrasound probe 1, a phantom 33 containing the dispersed fluid 31 was measured. The ultrasound probe 1 performed the measuring process by using a planar wave compound scan of which the transmission center frequency was 6 MHZ. FIGS. 4 and 5 illustrate a measurement cross-section 34 and a measurement cross-section 32, respectively. The dispersed fluid 31 was caused to flow as a pulsation flow by using a pump 30 and measured at pulsation cycles of 0.5 Hz and 1 Hz. FIGS. 6 and 7 each indicate an average frequency (a spatial distribution μ_{f}(x) of the frequency information) calculated by implementing the method according to the embodiment. The spatial distribution μ_{f}(x) of the frequency information is indicated in FIG. 6 with a pulsation cycle of the pulsation flow made by the pump 30 being 0.5 Hz and is indicated in FIG. 7 with a pulsation cycle made by the pump 30 being 1 Hz. The average frequency is positioned near the frequency 0.5 Hz in FIG. 6 and is positioned near the frequency 1 Hz in FIG. 7. It was thus confirmed that the method according to the embodiment is capable of generally correctly calculating the spatial distribution μ_{f}(x) of the frequency information.

As explained above, according to the first embodiment, the processing circuitry 18 is configured to calculate the periodicity information of the movements of the patient by employing the calculating function 18b and to generate the spatial distribution of the periodicity information by employing the generating function 18c. As a result, it is possible to realize the imaging of the periodicity of the chronological changes in the imaged target.

### Second Embodiment

In the first embodiment, the example was explained in which, at step S130, the time waveform of the Doppler velocity is extracted as the information s(t,x) related to the motion velocity of the target inside the patient. However, possible embodiments are not limited to this example. In a second embodiment, an example will be explained in which an echo signal intensity after the MTI filter is applied is extracted as information related to motion velocity of a target inside the patient.

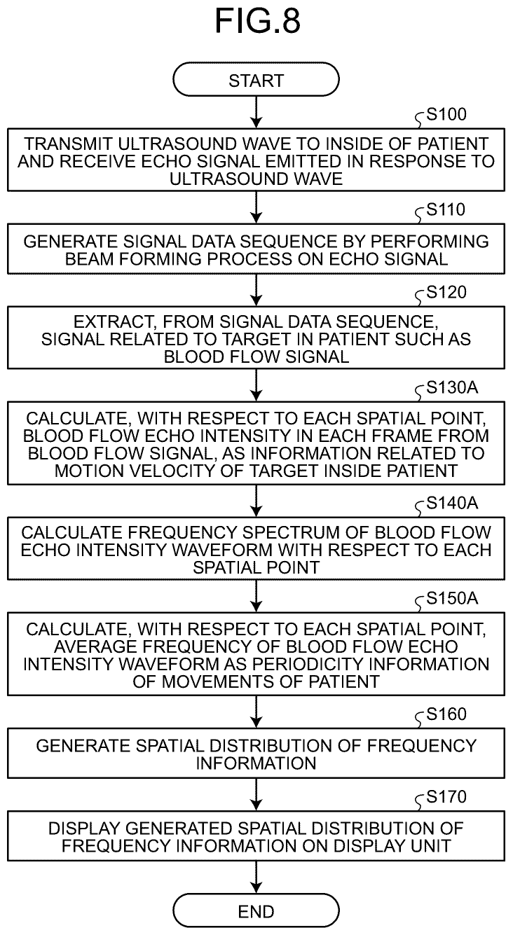

FIG. 8 is a flowchart illustrating a flow in processes performed by an ultrasound diagnosis apparatus according to the second embodiment. Because the processes other than the processes at steps S130A through S150A are the same as those in FIG. 3, duplicate explanations thereof will be omitted.

At step S130A, by employing the analyzing function 18a serving as an analyzing unit, the processing circuitry 18 extracts, with respect to each spatial point x, information s(t,x) that is related to the motion velocity of the target inside the patient and is included in the signal data sequence obtained from the echo signal, for each frame t. In this situation, in the second embodiment, by employing the analyzing function 18a, the processing circuitry 18 extracts, in place of the time waveform of the Doppler velocity, the echo signal intensity after the MTI filter is applied at step S120, as information related to motion intensity. By employing the analyzing function 18a, the processing circuitry 18 extracts, with respect to each frame t, the echo signal intensity from the data sequence obtained on the basis of the echo signal, as the information s(t,x) related to the motion velocity of the target inside the patient.

In relation to the above, a significance of extracting the echo signal intensity as the information s(t,x) related to the motion velocity of the target inside the patient will be explained. The echo signal intensity after the MTI filter is applied increases in proportion to blood flow velocity. Thus, by performing a frequency analysis on the echo signal intensity in place of the velocity waveform, it is possible to achieve similar advantageous effects as those achieved by performing the frequency analysis on the velocity waveform. In addition, the precision level of the echo signal intensity does not get degraded by aliasing. Thus, by analyzing the echo signal intensity, it is possible to inhibit the precision level degradation that may be caused by aliasing, in comparison to the example with the velocity waveform.

Subsequently, at step S140, by employing the calculating function 14b, the processing circuitry 18 calculates the frequency spectrum S(i,x) of the echo signal intensity waveform, with respect to each spatial point x. In an example, by employing the calculating function 14b, the processing circuitry 18 calculates the complex frequency spectrum S(i,x) with respect to a plurality values of the frequency f(i), by performing a Fourier analysis on the echo signal intensity, which is the information s(t,x) related to the motion velocity and extracted with respect to each spatial point x.

After that, at step S150, by employing the calculating function 14b serving as a calculating unit, the processing circuitry 18 calculates, with respect to each space, periodicity information of the movements of the patient, on the basis of the information related to the motion velocity of the target inside the patient. In an example, by employing the calculating function 14b, the processing circuitry 18 calculates, with respect to each spatial point, an average frequency of the echo signal intensity waveform, as the periodicity information of the movements of the patient.

In an example, by employing the calculating function 14b, the processing circuitry 18 calculates the average frequency μ_{f} as the periodicity information, by adding together values of the frequency f(i) while applying weights thereto with the use of the magnitudes of the frequency spectra S(i) calculated at step S140, according to Expression (1).

As explained above, according to the second embodiment, by employing the calculating function 18b, the processing circuitry 18 is configured to perform the frequency analysis by using the blood flow echo intensity in place of the blood flow velocity waveform and to generate the spatial distribution of the periodicity information. The second embodiment serves as an embodiment alternative to the first embodiment and is also able to inhibit the precision level degradation that may be caused by aliasing, by using the blood flow echo intensity in place of the velocity waveform.

### Third Embodiment

In the first embodiment, at step S150, the periodicity information of the movements of the patient is calculated by using the frequency spectra from the entire frequency range. In a third embodiment, by employing the calculating function 18b, the processing circuitry 18 is configured to calculate an average frequency in a limited frequency band, as the periodicity information. As a result, it is possible to enhance the precision level of the periodicity information to be calculated.

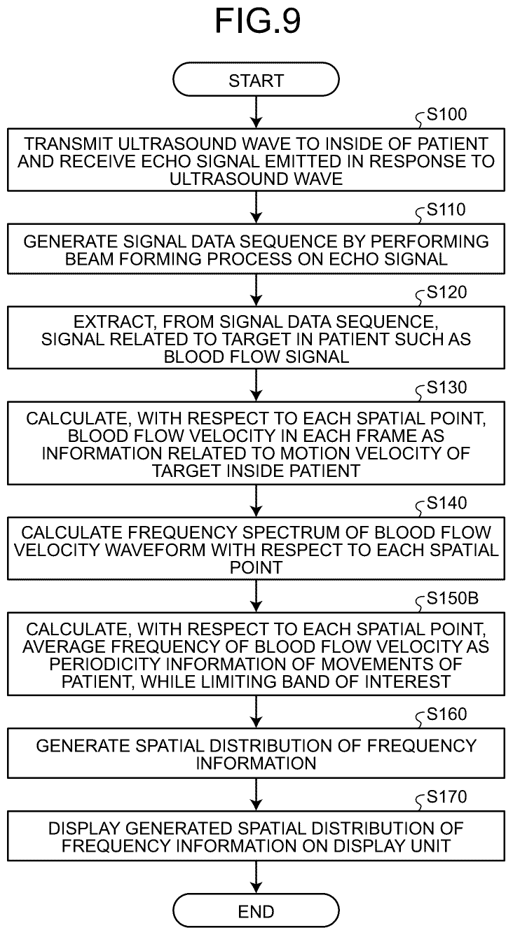

FIG. 9 illustrates a flow in processes performed by an ultrasound diagnosis apparatus according to the third embodiment. Because the processes other than the process at step S150B are the same as those in FIG. 3, duplicate explanations thereof will be omitted.

At step S150B, by employing the calculating function 14b serving as a calculating unit, the processing circuitry 18 calculates, with respect to each space, the periodicity information of the movements of the patient, on the basis of the information related to the motion velocity of the target inside the patient, while limiting a band of interest (frequencies). In an example, by employing the calculating function 14b, the processing circuitry 18 calculates, with respect to each spatial point, an average frequency related to chronological changes in the motion of the target in the limited frequency band, e.g., an average frequency of the blood flow velocity, as the periodicity information of the movements of the patient, while limiting the band of interest.

More specifically, by employing the calculating function 14b serving as a calculating unit, the processing circuitry 18 calculates an average frequency μ′ᵢ as the periodicity information, by adding together values of the frequency f(i) while applying weights thereto with the use of the magnitudes of the frequency spectra S(i) calculated at step S140. In an example, by employing the calculating function 14b, the processing circuitry 18 calculates the average frequency u′ᵢ as the periodicity information, by adding together the values of the frequency f(i) while applying the weights thereto with the use of the magnitudes of the frequency spectra S(i) calculated at step S140, according to Expression (2) presented below.

$${{μ}^{′}}_{f} = \frac{{\sum }_{i = {i}_{1}}^{{i}_{2}} f ( i ) \vert S ( i ) \vert}{{\sum }_{i = {i}_{1}}^{{i}_{2}} \vert S ( i ) \vert} \qquad ( 2 )$$

In the above expression, i₁ and i₂ denote indices corresponding to a lower limit frequency and an upper limit frequency of the band of interest. While i is an index indicating a frequency, the frequency band in the range of f(i₁) to f(i₂) inclusive corresponding to the values of i in the range of i₁ to i₂ inclusive serves as the limited frequency band to be used for the calculation of the average frequency.

In relation to the above, a reason for providing the lower limit frequency i₁ of the band of interest is that a stationary component of which the frequency is near 0 may be an obstacle in the calculation of the average frequency. In other words, by employing the calculating function 18b and calculating the chronological changes in the motion of the target, while limiting the frequency band to the frequency band equal to or larger than the lower limit frequency i₁, the processing circuitry 18 is able to enhance the precision level of the calculation of the average frequency.

Further, a reason for providing the upper limit frequency i₂ of the band of interest is that it is expected to be possible to enhance the precision level of the calculation of the average frequency, by eliminating signal components outside the frequency band of interest. In other words, a clinically important frequency band is the frequency band corresponding to a pulsation cycle, for example. Thus, as a result of the calculating function 14b eliminating frequency bands that greatly deviate from the frequency band corresponding to the pulsation cycle, from the calculation of the average frequency, the processing circuitry 18 is able to enhance the precision level of the calculation of the average frequency.

As explained above, according to the third embodiment, by employing the calculating function 18b, the processing circuitry 18 is configured to calculate the periodicity information of the movements of the patient, while limiting the band of interest. As a result, it is possible to calculate the periodicity information of the movements of the patient with a higher level of precision.

### Fourth Embodiment

In the embodiments described above, the periodicity information of the chronological changes in the motion of the target inside the patient is calculated; however, possible embodiments are not limited to those examples. In a fourth embodiment, by employing the calculating function 18b, the processing circuitry 18 is configured to further calculate information related to a stationary component among the chronological changes in the motion of the target inside the patient. As a result, regarding the target inside the patient, it is possible to perform an analysis also on the stationary component, in addition to the analysis related to the periodicity information. For example, by distinguishing a vein, which has a stationary flow, from an artery and extracting only the artery, it is possible to enhance the precision level of diagnosing processes.

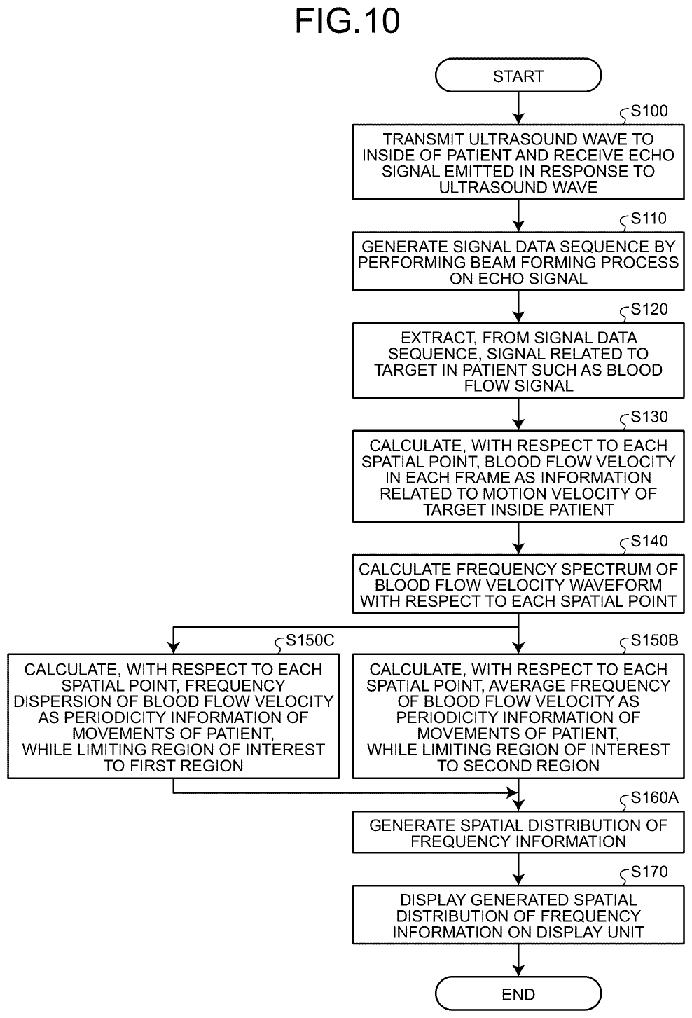

FIG. 10 is a flowchart illustrating a flow in processes performed by an ultrasound diagnosis apparatus according to the fourth embodiment. In this situation, because the processes other than the processes at steps S150B to S170 are the same as those in the first embodiment, duplicate explanations thereof will be omitted.

Similarly to the first embodiment, at step S140, by employing the calculating function 18b, the processing circuitry 18 calculates, with respect to each spatial point x, the frequency spectrum S(i,x) of the blood flow velocity waveform. After that, similarly to the third embodiment, at step S150B, by employing the calculating function 14b serving as a calculating unit, the processing circuitry 18 calculates, with respect to each space, the periodicity information of the movements of the patient on the basis of the information related to the motion velocity of the target inside the patient while limiting the band of interest.

In an example, by employing the calculating function 14b, the processing circuitry 18 calculates, with respect to each spatial point, an average frequency related to chronological changes in the motion of the target in the limited frequency band, e.g., an average frequency of the blood flow velocity, as the periodicity information of the movements of the patient, while limiting the band of interest to a second region. More specifically, by employing the calculating function 14b and using Expression (2), the processing circuitry 18 calculates the average frequency μ′₁ as the periodicity information, by adding together values of the frequency f(i) while applying weights thereto with the use of the magnitudes of the frequency spectra S(i) calculated at step S140, while limiting the frequency band to the second region being the frequency band between the lower limit frequency i₁ and the upper limit frequency i₂.

Additionally, by employing the calculating function 18b, the processing circuitry 18 further calculates, at step S150C, information related to a stationary component (a DC component) among the motion of the target inside the patient. More specifically, by employing the calculating function 18b, the processing circuitry 18 calculates, with respect to each spatial point, a frequency dispersion related to the motion of the target inside the patient as the periodicity information of the movements of the patient, while limiting the band of interest to a first region of which the frequency is in the range of 0 to the threshold frequency i inclusive. The frequency dispersion denotes a degree of concentration of signal intensities in the DC component. Thus, at step S150C, the processing circuitry 18 is able to evaluate the stationary component related to the motion of the target inside the patient, by employing the calculating function 18b.

As explained above, in the fourth embodiment, by employing the calculating function 18b, the processing circuitry is configured to calculate the periodicity information of the movements of the patient at step S150B and is also capable of evaluating the stationary component at step S150C. As a result, at the time of performing the blood flow imaging, for example, it is possible to separate an artery from a vein and to also display the periodicity information regarding the blood flows.

Subsequently, at step S160A, by employing the generating function 14c serving as a generating unit, the processing circuitry 18 generates a spatial distribution u′_{f}(x) of the frequency information, on the basis of the periodicity information of the movements of the patient such as the average frequency μ_{f} calculated at step S150B with respect to each spatial point x.

After that, at step S170, by employing the display controlling function 18d, the processing circuitry 18 causes the monitor 2 serving as a display unit to display the generated spatial distribution of the frequency information.

By employing the calculating function 18b, the processing circuitry 18 may be configured to further calculate an index indicating pulsatility of the blood flow. In an example, by employing the calculating function 18b, the processing circuitry 18 may be configured to further calculate a Pulsatility Index (PI) or a Resistance Index (RI) being an index for evaluating resistance of a peripheral blood vessel, as the index indicating the pulsatility of the blood flow. More specifically, by employing the calculating function 18b, the processing circuitry 18 may calculate the Pulsatility Index (PI) by dividing the difference between Peak Systolic Velocity (PSV) and End-Diastolic blood flow Velocity (EDV) by Time-Averaged Maximum blood flow Velocity (TAMV). Further, by employing the calculating function 18b, the processing circuitry 18 is also capable of calculating the Resistance Index (RI) by obtaining a value resulting from dividing the difference between Peak Systolic Velocity (PSV) and End-Diastolic blood flow Velocity (EDV) by the Peak Systolic Velocity (PSV).

At step S170, by employing the display controlling function 18d, the processing circuitry 18 may be configured to control the monitor 2 serving as a display unit so that the periodicity information is displayed while being superimposed on at least one type of information selected from among: the abovementioned index indicating the pulsatility of the blood flow; the information related to the stationary component among the chronological changes in the motion of the target that was calculated at step S150C; and the information related to the Doppler velocity calculated at step S130. In an example, by employing the display controlling function 18d, the processing circuitry 18 may be configured to control the monitor 2 serving as a display unit so that at least one type of information mentioned above is displayed by using a first color bar, while the periodicity information calculated at step S150B is displayed by using a second color bar.

According to at least one aspect of the embodiments described above, it is possible to realize the imaging process for the periodicity in the chronological changes in the imaged target.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
transmitter/receiver circuitry configured to transmit an ultrasound wave to an inside of an examined subject and to receive an echo signal emitted in response to the ultrasound wave; and
processing circuitry configured
to extract, with respect to each spatial point, information that is related to motion velocity of a target inside the examined subject and included in a signal data sequence obtained from the echo signal,
to calculate periodicity information of a movement of the examined subject on a basis of the information, and
to generate a spatial distribution of the periodicity information.

2. The ultrasound diagnosis apparatus according to claim 1, wherein, the processing circuitry is configured to apply a Moving Target Indicator (MTI) filter to the signal data sequence and to extract information related to a blood flow signal as the information related to the motion velocity.

3. The ultrasound diagnosis apparatus according to claim 1, wherein the processing circuitry is configured to extract a time waveform of Doppler velocity, as the information related to the motion velocity.

4. The ultrasound diagnosis apparatus according to claim 2, wherein the processing circuitry is configured to extract an echo signal intensity after the MTI filter is applied, as the information related to the motion velocity.

5. The ultrasound diagnosis apparatus according to claim 1, wherein the processing circuitry is configured to calculate an average frequency related to chronological changes in motion of the target, as the periodicity information.

6. The ultrasound diagnosis apparatus according to claim 5, wherein the processing circuitry is configured to calculate the average frequency in a limited frequency band, as the periodicity information.

7. The ultrasound diagnosis apparatus according to claim 5, wherein the processing circuitry is configured to calculate the average frequency as the periodicity information, by performing a Fourier analysis on the information related to the motion velocity extracted with respect to each spatial point, so as to calculate frequency spectra with respect to a plurality of frequencies, and further adding together the frequencies while applying weights thereto by using magnitudes of the frequency spectra.

8. The ultrasound diagnosis apparatus according to claim 1, wherein the processing circuitry is configured to further calculate information related to a stationary component among chronological changes in motion of the target.

9. The ultrasound diagnosis apparatus according to claim 1, wherein the processing circuitry is configured to further calculate one of a Pulsatility Index (PI) and a Resistance Index (RI), as an index indicating pulsatility of a blood flow.

10. The ultrasound diagnosis apparatus according to claim 1, further comprising: a display controlling unit configured to control a display unit so that the periodicity information is displayed while being superimposed on at least one type of information selected from among: an index indicating pulsatility of a blood flow; information related to a stationary component among chronological changes in motion of the target; and information related to Doppler velocity.

11. The ultrasound diagnosis apparatus according to claim 10, wherein the processing circuitry is configured to control the display unit so as to display said at least one type of information by using a first color bar and to display the periodicity information by using a second color bar.

12. The ultrasound diagnosis apparatus according to claim 1, wherein
the target is a region including an umbilical blood vessel, and
the processing circuitry is configured to calculate information corresponding to a heart rate of a mother as the periodicity information of the movement with respect to a region corresponding to a blood vessel of the mother and to calculate information corresponding to a heart rate of a fetus as the periodicity information of the movement with respect to a region corresponding to the umbilical blood vessel.

13. The ultrasound diagnosis apparatus according to claim 1, wherein
the target is a region including an intrarenal interlobar vein, and
the processing circuitry is configured to calculate periodicity information of a blood flow in the intrarenal interlobar vein and to generate information related to a state of a heart disease on a basis of the calculated periodicity information of the blood flow in the intrarenal interlobar vein.

14. The ultrasound diagnosis apparatus according to claim 1, wherein
the processing circuitry is configured to extract a signal included in the signal data sequence with respect to each of motion directions of the target, and
the processing circuitry is configured to generate the spatial distribution of the periodicity information with respect to each of the motion directions of the target.

15. An ultrasound diagnosis method comprising:
transmitting an ultrasound wave to an inside of an examined subject and receiving an echo signal emitted in response to the ultrasound wave;
extracting, with respect to each spatial point, information that is related to motion velocity of a target inside the examined subject and included in a signal data sequence obtained from the echo signal;
calculating periodicity information of a movement of the examined subject on a basis of the information; and
generating a spatial distribution of the periodicity information.

16. An image processing apparatus comprising processing circuitry configured:
to extract, with respect to each spatial point, information that is related to motion velocity of a target inside an examined subject and included in a signal data sequence obtained from an echo signal emitted in response to an ultrasound wave transmitted to an inside of the examined subject;
to calculate periodicity information of a movement of the examined subject on a basis of the information; and
to generate a spatial distribution of the periodicity information.
